(19) United States
(12) Patent Application Publication
(10) Pub. No.: US 2019/0209050 A1
(43) Pub. Date: Jul. 11, 2019

(54) WEARABLE DEVICE AND METHOD OF USING SAME TO MONITOR MOTION STATE

(71) Applicant: Goertek Inc., Shandong Province (CN)

(72) Inventors: Pengcheng SU, Shandong Province (CN); Yifan ZHANG, Shandong Province (CN)

(21) Appl. No.: 16/327,173

(22) PCT Filed: Jul. 13, 2017
(86) PCT No.: PCT/CN2017/092739

§ 371 Date: Feb. 21, 2019

(30) Foreign Application Priority Data
Aug. 31, 2016 (CN) 201610789754.9

(51) Int. Cl. A61B 5/11; A61B 5/00; A63B 24/00

(52) U.S. Cl. CPC A61B 5/1123; A61B 5/1121; A61B 5/7267; A61B 5/7246; A63B 24/0062; A63B 2244/20; A61B 2562/0219; A63B 2024/0068; A63B 2220/836; A63B 2220/44; A61B 2503/10

(57) ABSTRACT

A wearable device and a method for monitoring a movement state by using the same are disclosed. A sensor is provided in the wearable device. The method comprises: controlling the sensor to collect movement data of a user when a monitoring process starts; extracting one or more features for identifying the movement state of the user from the movement data to obtain test data; and matching the test data with stored template data representing predetermined movement states, to obtain template data that successfully match the test data, and determining occurrence of a movement state corresponding to the matched template data associated with the test data.

### CROSS REFERENCE TO RELATED APPLICATIONS

This application is a U.S. National Stage entry under 35 U.S.C. § 371 based on International Application No. PCT/CN2017/092739, filed on Jul. 13, 2017, which was published under PCT Article 21(2) and which claims priority to Chinese Patent Application No. 201610789754.9, filed on Aug. 31, 2016. These applications are hereby incorporated herein in their entirety by reference.

### TECHNICAL FIELD

This application pertains to the field of wearable devices, and more particularly to a wearable device and a method for monitoring a movement state by using the same.

### BACKGROUND ART

Conventionally, the monitoring and tracking of the movement state (for example, the swimming state) of the athlete or exerciser is mainly performed visually, and the video data are analyzed and identified afterwards, so the statistic and identifying results cannot be given in real time. Although there have been some professional sports monitoring equipment which can analyze movement data such as swimming strokes and exercise amount, they are expensive and inconvenient to carry, and not suitable for ordinary swimmers. Moreover, the accuracy of the movement state monitoring results obtained by conventional movement state monitoring methods also need improvement. In addition, other objects, desirable features and characteristics will become apparent from the subsequent summary and detailed description, and the appended claims, taken in conjunction with the accompanying drawings and this background.

### SUMMARY

The present disclosure provides a wearable device and a method for monitoring a movement state by using the same, so as to solve the problems of the prior art that the movement state of an ordinary athlete or exerciser can only be monitored and identified afterwards, the portability of the monitoring equipment is poor, the accuracy of the monitoring result is low, and the need of ordinary swimmers for monitoring the swimming state cannot be satisfied.

According to an aspect of the present disclosure, a method for monitoring a movement state by using a wearable device is provided, wherein a sensor is provided in the wearable device, and the method comprises:

controlling the sensor to collect movement data of a user when a monitoring process starts;

extracting one or more features for identifying the movement state of the user from the movement data to obtain test data; and

matching the test data with stored template data representing predetermined movement states, to obtain template data that successfully match the test data, and determining occurrence of a movement state corresponding to the matched template data associated with the test data.

According to another aspect of the present disclosure, a wearable device is provided, wherein a sensor is provided in the wearable device, and the wearable device comprises:

a data collecting unit, for controlling the sensor to collect movement data of a user when a monitoring process starts;

a feature extracting unit, for extracting one or more features for identifying the movement state of the user from the movement data to obtain test data; and

a state monitoring unit, for matching the test data with stored template data representing predetermined movement states, to obtain template data that successfully match the test data, and determining occurrence of a movement state corresponding to the matched template data associated with the test data.

The advantageous effects of the present disclosure are as follows. The method for monitoring a movement state by using a wearable device according to embodiments of the present disclosure takes advantage that the wearable device is programmable and can be simultaneously embedded with a plurality of low-cost micro-electromechanical system (MEMS) sensors (for example, accelerometers, gyroscopes), which provide hardware and software support for the sensor-based identification of the movement state such as swimming. Moreover, wearable devices are typically light and compact, and are convenient for users to carry with them, so they can identify the movement state at any time during swimming and other exercises, and perform corresponding statistics of exercise amount to give users corresponding feedback, which helps people improve their exercise.

Compared with the prior art, the identification of the movement state (for example, swimming strokes) based on the movement sensor in the wearable device is flexible and reliable, and is not affected by the environment, light, etc.; the system is simple and easy to implement; it is convenient for the user to carry and can satisfy the needs of ordinary athletes and exercisers, and thus the market competitiveness of the wearable device is also enhanced.

Furthermore, by adopting a technical means combining the feature extraction and template data matching, the technical solutions of the present disclosure can identify a plurality of movement states of the user, and it is proved by experimentation that the accuracy of the monitoring results obtained by the technical solutions of the present disclosure is high.

### BRIEF DESCRIPTION OF DRAWINGS

The present invention will hereinafter be described in conjunction with the following drawing figures, wherein like numerals denote like elements, and:

FIG. 1 is a flow chart of a method for monitoring a movement state by using a wearable device according to an embodiment of the present disclosure;

FIG. 2 is a flow chart of a method for monitoring the state by using a wearable device according to another embodiment of the present disclosure;

FIG. 3 is a schematic diagram of data collecting according to still another embodiment of the present disclosure;

FIG. 4 is a schematic diagram of data windowing processing according to still another embodiment of the present disclosure;

FIG. 5 is a structural block diagram of a wearable device according to an embodiment of the present disclosure; and

FIG. 6 is a schematic diagram of the hardware structure of a wearable device according to still another embodiment of the present disclosure.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description.

At present, the thriving of wearable devices such as smart watches has made the wide use of movement identification possible. First, wearable devices such as smart watches are programmable, and can be simultaneously embedded with a plurality of low-cost MEMS sensors such as accelerometers and gyroscopes, which provide hardware and software support for the sensor-based identification of the movement state such as swimming. Second, the smart watch is light and compact, and convenient for the user to wear at any time, so it can identify the strokes of exercise at any time during swimming and other exercises, and perform corresponding statistics of exercise amount to give users corresponding feedback, which helps people improve their exercise.

Based on this, the inventive concept of the present disclosure is to realize the detection of the movement state by using the wearable device, and more specifically, compare the test data collected by the movement sensor in the wearable device such as the smart watch with the preset template data, to obtain the identifying results. Compared with the conventional methods, this method for monitoring the movement state is flexible and reliable, and is not affected by factors such as environment and light, the system is simple and easy to implement; and it is convenient for the user to carry.

### First Embodiment

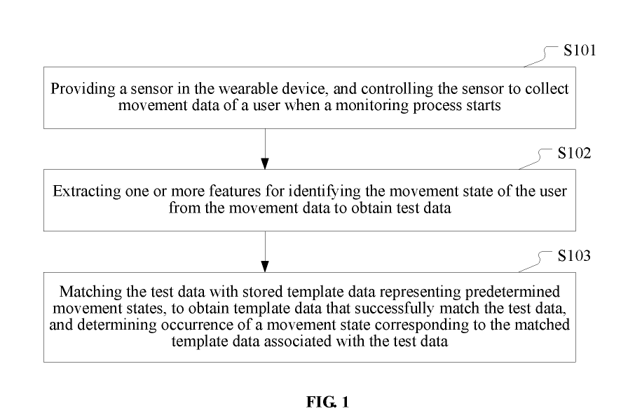

FIG. 1 is a flow chart of a method for monitoring a movement state by using a wearable device according to an embodiment of the present disclosure. Referring to FIG. 1, a sensor is provided in the wearable device, and the method comprises the following steps:

Step S101, controlling the sensor to collect movement data of a user when a monitoring process starts;

Step S102, extracting one or more features for identifying the movement state of the user from the movement data, to obtain test data; and

Step S103, matching the test data with stored template data representing predetermined movement states, to obtain template data that successfully match the test data, and determining occurrence of a movement state corresponding to the matched template data associated with the test data.

The above steps S101 to S103 may be implemented by a function module provided in the wearable device, such as processor.

As can be seen from FIG. 1, the method for monitoring a movement state by using a wearable device according to the present embodiment, the sensor is controlled to collect movement data of a user; one or more features for identifying the movement state of the user are extracted from the movement data to obtain test data; then the test data is matched with the stored template data representing the predetermined movement states, and after the template data that successfully match the test data is obtained, it is determined that the movement state corresponding to the template data that is obtained by matching and is associated with the test data occurs. In this way, by taking advantage of the wearable characteristic of the wearable device, the movement state of the athlete or exerciser can be monitored and identified in real time, so it is convenient for the athlete or exerciser to know his or her movement state, which helps the athlete or exerciser improve the exercise effect. In addition, the wearable devices are easier to use and less expensive than professional sports monitoring equipment, so they can satisfy the needs of ordinary athletes and exercisers for monitoring the movement state.

In the following embodiments, the implementation of the method for monitoring the movement state by using a wearable device according to the present disclosure will be described by taking the monitoring of swimming movement state as an example. It should be emphasized that the technical solutions of the present disclosure can also be applied in identifying other movement states such as walking, running, going upstairs, and going downstairs. The specific implementation of the monitoring of other movement states may be referred to the description of the monitoring of swimming movement state in the following embodiments. In addition, in the description of the following embodiments, a smart watch is taken as an example of the wearable device, and the smart watch is used to monitor the swimming movement state of the swimmer.

There are still many problems to be solved when a smart watch is used in practice to monitor and identify the movement state such as swimming. Exemplarily,

(1) The swimming strokes and habits of different users in swimming are different and even very different. The water resistance and fluctuation in the swimming pool also have a great influence on the sensor readings. Thus, it is necessary to adopt reasonable technical means to extract effective features, and use an appropriate identifying method to achieve high identification ability for different users.

(2) The identification of exercise strokes using a smart watch must consider the amount of calculation and power consumption. The power of a wearable device such as a smart watch is fairly limited. In the identifying process, the continuous sensing of the smart watch will consume a lot of energy. Thus, it is necessary to adopt effective strategies to control the complexity of algorithm, reduce the amount of calculation, and improve the sensing efficiency, thereby improving the user-friendliness.

In view of these problems, the present disclosure adopts simple and effective preprocessing measures to remove the influence of noise, extracts a limited quantity of time domain features that are truly distinguishable, to avoid complicated feature calculations and reduce the amount of calculation.

In addition, the support vector machine (SVM) is used to identify swimming strokes. The SVM is suitable for small sample training sets, and does not need a complicated training process. Moreover, it has excellent generalization ability, and can well identify the swimming strokes of different users. Meanwhile, the classifier generated after the SVM is trained is simple. Compared with the identification algorithms such as KNN (k-Nearest Neighbor), it needs only a small amount of sample information, and saves the storage space of the template data. KNN algorithm is one of the simplest algorithms in data mining classification technology.

### Second Embodiment

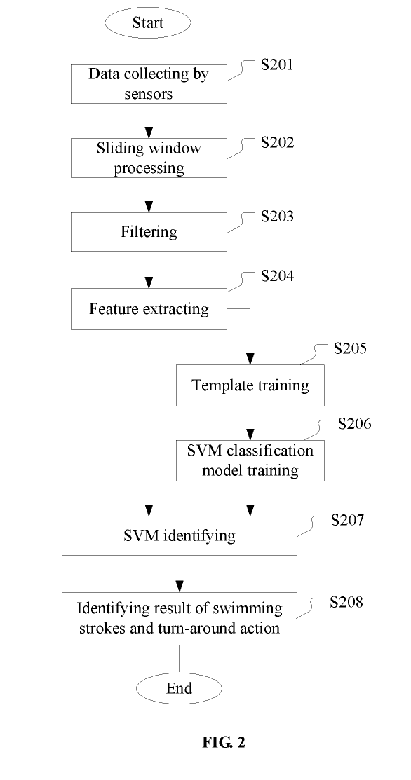

FIG. 2 is a flow chart of a method for monitoring a movement state by using a wearable device according to another embodiment of the present disclosure. Referring to FIG. 2, the overall process of the method for monitoring a movement state by using a wearable device is as follows.

Firstly, four basic swimming strokes are identified, and the occurrence of the turn-back action may also be identified. Here, the movement state is monitored and identified mainly based on time domain features and support vector machine (SVM).

Secondly, for the three-axis acceleration data collected during swimming, the sliding window processing method is adopted, and there should be a certain overlap between adjoining sliding windows. For the three-axis acceleration data of X, Y and Z axes in the sliding windows, sliding mean filtering is performed respectively to remove the influence of noise, and relatively smooth data are obtained.

Then, the time domain features are extracted respectively in each of the sliding windows.

Finally, the support vector machine (SVM) method is used to classify and identify. Before starting to identify, swimming data of several different standard swimming strokes are collected for training, and a SVM multi-class classifier is obtained. In the process of actual applications, for the acceleration data generated during the swimming process of the user, the trained SVM classifier is used for effective identification.

Exemplarily, referring to FIG. 2, the method includes the following steps:

The process starts, and step S201 is executed.

Step S201, data collecting by sensors.

In the present embodiment, an acceleration sensor is used to identify four basic swimming strokes, and the occurrence of the turn-back action in swimming may also be identified. Where, turn-back action refers to the movement in which an athlete swims to the end of the pool, turns back and continues swimming.

In step S201, the sensor is controlled to collect movement data of the user of one or more axis directions. When a monitoring process starts, a three-axis acceleration sensor is controlled to collect three-axis acceleration data of the user when he/she is swimming, and the collected three-axis acceleration data is saved in a buffer.

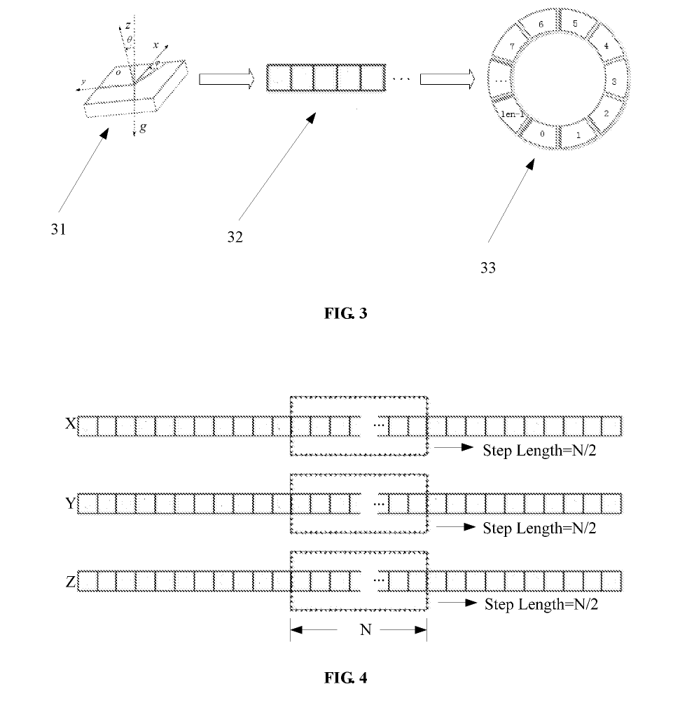

FIG. 3 is a schematic diagram of data collecting according to still another embodiment of the present disclosure. Referring to FIG. 3, 31 is a three-axis acceleration sensor, 32 is the collected acceleration data, 33 is a ring buffer. The three-axis acceleration sensor 31 collects a human body action and the three-axis acceleration data 32 are obtained. The collected three-axis acceleration data 32 is put into a corresponding ring buffer 33 (one ring buffer 33 is shown in FIG. 3). By using the ring buffer 33, the design of the present embodiment can save the storage space of the system, and facilitate subsequent sampling of the collected acceleration data and the subsequent sliding windowing processing.

It can be understood by those skilled in the art that in other embodiments of the present disclosure, the collected acceleration data 32 may not be put in the ring buffer 33, which is not limited herein.

In addition, it should be emphasized that FIG. 3 is only a schematic illustration by taking the collecting of the three-axis acceleration of a human body action by using an acceleration sensor as an example. However, in other embodiments of the present disclosure, the three-axis angular velocity data of the human body action may also be collected by using a gyroscope. Alternatively, the three-axis acceleration data are collected by using the acceleration sensor and at the same time the three-axis angular velocity data are collected by using the gyroscope, and then the acceleration data and the angular velocity data are trained respectively, which is not limited herein.

In the method of the present embodiment, after the raw acceleration data are collected, and before one or more features for identifying the swimming state of the user are extracted from the swimming action data, the collected three-axis acceleration data are pre-processed.

Exemplarily, after step S201 is executed, step S202 is executed to perform a windowing preprocessing operation on the acceleration data.

Step S202, sliding window processing.

For the three-axis acceleration data collected during swimming, the sliding window processing method is used to sample simultaneously from the buffer according to a preset frequency, and a windowing processing is performed on the sampled data by using a sliding window of a preset step length to obtain acceleration data of each axis direction having a predetermined length. The moving step length of the sliding window satisfies the condition that data of adjoining sliding windows partially overlap. In other words, a certain overlap must be ensured between the adjoining sliding windows. The reason that the data between adjoining sliding windows are set to partially overlap is to prevent inaccurate identification caused by data omission.

FIG. 4 is a schematic diagram of a sliding windowing processing according to still another embodiment of the present disclosure. As shown in FIG. 4, the ring buffers that store the three-axis acceleration data of the X axis, the Y axis, and the Z axis respectively are sampled according to a preset frequency. A windowing processing is performed on the sampled data. In the present embodiment, the sampling frequency is 50 Hz (i.e., 50 data are sampled in one second), the length of each sliding window is N=50*T sampled data, where T is the length of time covered by the sliding window (i.e., seconds), and the moving step length of the sliding window is N/2 sampled data. The size of the sliding window is the length of the raw sampled data obtained within T seconds, i.e., N sampled data are taken out from the three ring buffers of the X axis, the Y axis, and the Z axis at the same time for test and identification.

It should be noted that, in the present embodiment, the moving step length of the sliding window is half of the size of the sliding window. It is understandable that in other embodiments of the present disclosure, the moving step length of the sliding window may be ⅓ or other ratio of the size of the sliding window, as long as it is ensured that the data in the adjoining sliding windows partially overlap. In addition, in the present embodiment, the window function used in the data windowing process is a rectangular window, which is a zero-power window of the time variable. However, in the present disclosure, the window function is not limited to the rectangular window, other window functions may be used, and there is no limitation on the window function.

Step S203, filtering.

Smoothing filtering is performed on the obtained acceleration data of each axis direction having the predetermined length by using K time-nearest neighbor mean filtering respectively, to remove interference noise. Exemplarily, for the X, Y and Z three-axis acceleration data in the sliding window, sliding mean filtering (for example, K time-nearest neighbor mean filtering) is performed to remove the influence of noise, and relatively smooth data are obtained.

In the present embodiment, performing filtering on the acceleration data of the predetermined length to filter out the interference noise comprises: with respect to a data point to be filtered of each axis direction of the raw data having the predetermined length, selecting a predetermined quantity of data points adjacent to the left side of the data point and a predetermined quantity of data points adjacent to the right side of the data point, calculating the mean value of the selected data points, and the value of the data points to be filtered is replaced by the mean value.

In other words, in the present embodiment, the K time-nearest neighbor mean filtering is used to perform filtering. In the K time-nearest neighbor mean filtering, the quantity K of the nearest neighbors in time is set in advance, and then in acceleration data of each axis, the mean value of the data consisting of K nearest neighbor data points to the left of any data point and K nearest neighbor data points to the right of this data point is taken as the value of this data point after filtered.

For the first K data points and the last K data points in the time series, special processing is required, and as many neighbor data points as possible should be taken as the objects of the averaging process.

Taking the data of the X axis in the three-axis acceleration data as an example, the K time-nearest neighbor mean filtering is:

$${a}_{xi}^{′} = \{ \begin{matrix}\frac{1}{i + K} {\sum }_{j = 1}^{i + K} {a}_{xj} & i \leq K \\ \frac{1}{N - i + K + 1} {\sum }_{j = i - K}^{N} {a}_{xj} & i \geq N - K + 1 \\ \frac{1}{2 K + 1} {\sum }_{j = i - K}^{i + K} {a}_{xj} & K < i < N - K + 1\end{matrix} \qquad formula ( 1 )$$

where N is the length of the X axis data, i.e., the size of the sliding window (in the present embodiment, the data length is 50); K is the quantity of neighbors selected in advance, i.e., the selected quantity of the nearest neighbors respectively to the left and right of a certain data point; αₓⱼ is the component on the X axis of the acceleration signal αⱼ, α′ₓᵢ is the data after filtered corresponding to αₓⱼ, i is the position index of the acceleration data on the X axis, j is the position index of the acceleration data on the X axis, and there is an auxiliary relationship between i and j.

It should be noted that, besides K time-nearest neighbor mean filtering, in other embodiments of the present disclosure, other filtering methods may be used, for example, median filtering and Butterworth filtering, as long as the raw acceleration data can be filtered. The filtering algorithm is not limited herein.

Step S204, feature extracting.

In the present embodiment, several features for identifying the swimming state of the user are extracted from the swimming action data after filtered, and the test data is obtained. Exemplarily, in the present embodiment, several of the following time domain features are extracted from the movement data of each axis direction: mean value, standard deviation, minimum, maximum, skewness, kurtosis and correlation coefficient.

For example, the following seven time domain features are extracted for each of the X, Y, and Z axes in the sliding window: mean value, standard deviation, minimum, maximum, skewness, kurtosis and correlation coefficient. Preferably, the seven time domain features are extracted from the X, Y, and Z axes to form a 21-dimensional feature vector.

It should be noted that, providing that the required identifying performance can be satisfied, in the feature extraction, the seven time domain features of only one axis direction (for example, the X axis direction, or the Y axis direction, or the Z axis direction) may be extracted, or several of the seven time domain features of only one axis direction may be extracted. For example, four time domain features, i.e., mean value, minimum, maximum, and skewness of the data on the X axis may be extracted. Alternatively, several of the seven time domain features of three axis directions may be extracted to form a feature vector. For example, four time domain features, i.e., mean value, minimum, maximum, and skewness of the data on the X axis, the Y axis, and the Z axis may be extracted, respectively. It is not limited herein.

Further, after the features are extracted, in order to improve the data operation speed and reduce the calculating complexity, the method of the present embodiment further comprises: calculating the correlation between the test data consisting of one or more features and the movement state of the user by using statistical analysis, screening the test data according to the correlation between the test data and the movement state of the user, to obtain test data after screened, and then matching the test data after screened with the template data.

Step S205, template training.

the template data are generated by using collected standard swimming state data of a plurality of users and stored in the smart watch. The standard swimming state data include at least the following types of data: breaststroke data, freestyle data, butterfly data, backstroke data, and turn-back state data. The step of identifying the swimming state of the user as a swimming state corresponding to the template data associated with the test data comprises: identifying the swimming state of the user as breaststroke swimming stroke, freestyle swimming stroke, butterfly swimming stroke, backstroke swimming stroke or turn-back state corresponding to the template data associated with the test data.

Step S206, SVM classification model training.

The SVM classifier is trained by using the template data. Any two types of template data are selected from the template data to train an SVM two-class classifier, and a trained SVM two-class classifier which can distinguish any two types of template data in N types of template data is obtained. The test data is matched with each trained SVM two-class classifier respectively to obtain matching results between the test data and each SVM two-class classifier. Each matching result corresponds to one piece of the template data. The number of occurrences of the template data is counted, and the template data with the most quantity of occurrences is taken as the template data that successfully match the test data.

It should be noted that, the template trained in step S205 and the SVM classification model trained in step S206 may be trained in advance and stored in the smart watch, so that during the process of identifying the swimming state by using the smart watch by the user, the template and the SVM classification model do not need to be trained anymore to save the time taken by swimming state identifying. In other words, in actual applications, step S205 and step S206 may be omitted.

Step S207, SVM identifying.

When the user's swimming stroke is being identified, the collected three-axis acceleration sensor data are processed similarly. After the test data of each sliding window are extracted, the swimming stroke currently used by the user can be identified by using the trained SVM two-class classifier.

Step S208, identifying result of swimming strokes and turn-back action.

The swimming state currently adopted by the user is determined according to the identifying result of step S207. If the position of the turn-back point of the user in swimming is identified, the quantity of the trips that the user has swum can be further counted, and parameters such as the speed of swimming can be further calculated according to the length of the pool.

In addition, the method in the present embodiment further comprises: after determining that the current swimming state of the user is the turn-back state, judging whether a time interval between a time point of occurrence of a current turn-back state and a time point of occurrence of a previous turn-back state is greater than a preset time threshold, and if yes, determining the current turn-back state to be judged as valid, if not, determining the current turn-back state to be judged as invalid; and when the turn-back state is judged as valid, saving the time point of occurrence of the current turn-back state, and updating the stored time point of occurrence of turn-back state with the time point of occurrence of the current turn-back state.

At this point, the flow process ends.

It can be seen from the above that compared with conventional methods such as using video analysis or professional detection equipment, if the acceleration sensor built in the smart watch is used, it is convenient to carry, flexible to use, and can identify swimming strokes in real time, and give the user real-time feedbacks, which makes it convenient for users to know his movement state and statistics at any time.

Moreover, in order to adapt to the limited source of a wearable device such as a smart watch, only a limited quantity of typical and truly distinguishable time domain features are extracted. Compared with extracting other frequency domain or time-frequency domain features, complicated feature calculations can be avoided, and the amount of calculation can be reduced.

In addition, the use of support vector machine (SVM) with good generalization ability in identification can provide non-user-restricted identifying ability. In other words, the swimming strokes of different users can be well identified, and the individual training for every user before use is not needed anymore, which facilitates the use and improves the user experience.

The following is a more detailed description of monitoring the swimming movement state of the user by using a smart watch.

### Third Embodiment

The method for monitoring the swimming movement state by using a smart watch according to the present embodiment specifically comprises the following steps:

Step 31, data collecting by sensors.

The X, Y and Z three-axis accelerations of the swimming action are collected by using a built-in acceleration sensor of the smart watch. The collected data are stored in ring buffers of a length Len respectively, as shown in FIG. 3.

Step 32, sliding window processing.

The three-axis acceleration data are taken out from the ring buffer to add a sliding window respectively, as shown in FIG. 4. Each window has a size of N samples, the length of time covered is T seconds, and the moving step length of the sliding window is step samples (for example, step=N/2, i.e., the data of adjoining windows overlap by half a window length). Subsequent filtering will be performed on the data of each window.

The purpose of the sliding window processing is as follows. In the sliding window processing, one data segment of a fixed length is taken from the sensor data each time, which may be understood as covering the sensor data with a sliding window. There is a certain length of overlap between adjoining sliding windows, such as an overlap of half a window length. Every time the data in the current sliding window is processed, including identifying after the feature extraction, the swimming stroke corresponding to the current time period can be determined. Thus, by using a sliding window of a fixed length, the processing becomes relatively simple, and moreover, features are extracted for the same length of data each time, which facilitates the next identification.

Step 33, filtering.

The filtering is performed on the collected raw acceleration data to filter out interference noise.

In an embodiment of the present disclosure, the K time-nearest neighbor mean filter is used for processing. In the K time-nearest neighbor mean filtering, the quantity K of the nearest neighbors in time is set in advance, and then in the acceleration data of each axis direction, the mean value of the sequence consisting of K elements to the left and K elements to the right of any point is taken as the value of this point after pre-processed. For the first K data points and the last K data points in the time series, special processing is required, and as many neighbors as possible should be taken as the objects of the averaging process.

Step 34, feature extracting.

On the basis of the preprocessing of the acceleration data, features are extracted from the acceleration data. Due to the complexity of extracting frequency domain and time-frequency domain features (for example, wavelet features), calculating these features on a smart watch is costly and time-consuming, which adversely affects the real-time identification of swimming strokes on a smart watch.

In view of this problem, in an embodiment of the present disclosure, only a limited quantity of truly distinguishable time domain features are extracted to avoid complicated feature calculations and reduce the amount of calculation.

Exemplarily, in an embodiment of the present disclosure, for the X, Y and Z three-axis acceleration data in each sliding window, the following seven time domain features are respectively extracted: mean value, standard deviation, minimum, maximum, skewness, kurtosis and correlation coefficients, which form a 21-dimensional feature vector.

Taking the X axis acceleration data xi (i=1, 2, . . . , N) as an example, the specific calculation methods of each time domain feature are as follows. The i in the following formulas (2) to (8) represents the position index of the acceleration data on the X axis; for example, i=5 indicates that the datum is the fifth datum in the acceleration data sequence on the X axis.

(1) Mean value:

$$\overline{x} = \frac{1}{N} {\sum }_{i = 1}^{N} {x}_{i} \qquad formula ( 2 )$$

(2) Standard deviation:

$${σ}_{x} = \sqrt{\frac{1}{N} {\sum }_{i = 1}^{N} {( {x}_{i} - \overline{x} )}^{2}} \qquad formula ( 3 )$$

where X̅ is the mean value of the X axis acceleration samples (data).

The standard deviation reflects the degree of discreteness of the acceleration data, and is also an important feature for identifying static actions and dynamic actions. The standard deviation may be used to determine whether the user is currently in a relatively static state. If the standard deviations of the acceleration data in the three axis directions are all less than a preset threshold, the user will be considered not currently swimming, and no further identifying will be performed.

(3) Minimum and maximum:

min_x=min({xᵢ,i=1,2, . . . ,N}) formula (4)

max_x=max({xᵢ,i=1,2, . . . ,N}) formula (5)

(4) Skewness:

$${SK}_{x} = \frac{N {\sum }_{i = 1}^{N} {( {x}_{i} - \overline{x} )}^{3}}{( N - 1 ) ( N - 2 ) {σ}_{x}^{3}} \qquad formula ( 6 )$$

where X̅ is the mean value of the X axis acceleration samples (data), and σₓ is the standard deviation of the X axis acceleration samples (data).

Skewness is a statistical feature used to measure the direction and degree of skewness of the acceleration data distribution.

(5) Kurtosis:

$${K}_{x} = \frac{{\sum }_{i = 1}^{N} {( {x}_{i} - \overline{x} )}^{4} {f}_{i}}{N {σ}_{x}^{4}} \qquad formula ( 7 )$$

where X̅ is the mean value of the X axis acceleration samples (data), σₓ is the standard deviation of the X axis acceleration samples (data), and fᵢ is the sampling interval of the acceleration samples (data).

Kurtosis reflects the steepness of the acceleration data at the peak of the data curve and is an important statistical feature.

(6) Correlation coefficient:

$${r}_{yz} = \frac{{\sum }_{i = 1}^{N} ( {y}_{i} - \overline{y} ) ( {z}_{i} - \overline{z} )}{\sqrt{{\sum }_{i = 1}^{N} {( {y}_{i} - \overline{y} )}^{2}} \sqrt{{\sum }_{i = 1}^{N} {( {z}_{i} - \overline{z} )}^{2}}} \qquad formula ( 8 )$$

where y̅ and z̅ are the mean value of the acceleration samples (data) on the X and Y axes, respectively.

Correlation coefficient is an indicator of the degree of linear correlation between variables.

Similarly, the seven time domain features, namely mean value, standard deviation, minimum, maximum, skewness, kurtosis and correlation coefficient, may also be calculated for the data on the Y axis and the Z axis. Thus, for each sliding window, the time domain features extracted from the X, Y and Z three-axis data can form a 21-dimensional feature vector.

These features are calculated directly in the time domain, so a complicated transform coefficient processing is not required. Other relatively complicated algorithms such as Fourier transform FFT and wavelet transform need transform coefficients. If these algorithms that need transform coefficients are used, the processing and extraction of the data of each sliding window requires a larger amount of calculation. According to the test results for the swimming scenes, the time domain features used in the present disclosure are all important statistical features, and have sufficient distinguishing ability for swimming strokes. Compared with these time domain features, when FFT, wavelet transform and other feature extracting methods are used to identify, the identifying performance is not noticeably improved, but the amount of calculation is increased.

In order to further reduce the amount of calculation, when the template is trained, it is advisable to directly select several combinations of the seven features for identification, and select the combination with the best performance according to the test results, providing that the identifying performance is not noticeably reduced. Alternatively, after extracting the seven time domain features of the three axis directions respectively and obtaining a total 21-dimensional feature vector, a feature selection method may be used to further reduce the dimension of the feature vector, providing that the identifying performance is not reduced. The feature selection method may be a principal component analysis (PCA), a linear discriminant analysis (LDA), or a boosting algorithm, etc.

Step 35, SVM Classifier Training

Support vector machine (SVM) is suitable for small sample training sets, and has excellent generalization and promotion ability as well as good non-user dependence, so it can well identify swimming strokes of different users. Meanwhile, the classifier generated after the SVM is trained is simple, and compared with identification methods such as KNN, it only needs a small amount of sample information, which saves the storage space of the template. Therefore, in the present embodiment, the SVM classifier is used to identify swimming strokes.

Moreover, compared with DTW (Dynamic Time Warping), SVM classification and identification may use a sliding window of a fixed length, so the processing is relatively simple, and it is not necessary to calculate the start time and end time of the user action, and thus the processing speed is improved.

Assuming the sample set is (sᵢ, tᵢ), i=1, 2, . . . , n, where sᵢ is the sample point and tᵢ ∈{−1, +1} is the corresponding type label, the optimal classification function is expressed as:

$$f ( s ) = sgn ( {\sum }_{i = 1}^{n} {α}_{i}^{*} {t}_{i} K ( s , {s}_{i} ) + {b}^{*} ) \qquad formula ( 9 )$$

where sgn ( ) is a sign function, αᵢ^{*} and b* are the optimal solutions obtained when the solving classification interval is maximum and when the correct classification is ensured; K(s, sᵢ) is the kernel function, which corresponds to the inner product operations in transformation space.

When the swimming state is identified, the test data to be identified is substituted into the above formula (9) for calculation, and the type of the test data can be judged according to the sign outputted by the sign function.

Obtaining the SVM classifier in step 35 comprises two parts: training and using (for identification). Exemplarily, the training of the SVM classifier comprises:

In the SVM classification model (see formula 9 above), a radial basis function (RBF) may be selected as the kernel function. For RBF, the parameters to be determined are the kernel function parameter γ and the penalty factor C. In order to determine the optimal parameters C and γ to improve the identifying accuracy of the SVM classifier, the present disclosure employs a cross-validation based grid search method. In other words, different pairs of parameters (C, γ) are searched, and the pair of parameters with the highest accuracy is selected as the optimal result by using the cross-validation method.

In training the classifier, first, the acceleration data of the four basic swimming strokes of P different users are sampled, and processed according to steps S201-S204 shown in FIG. 2 respectively. Then, for each person, M (if a three-axis acceleration sensor is used, M=3) feature vectors of the sliding window are intercepted, and finally a feature matrix of 4*P*M rows and 21 columns is obtained, which is used to train and construct the SVM multi-class classifier.

The classification and identification using the trained SVM classifier comprises:

When the user's swimming stroke is identified, the collected three-axis acceleration data are processed according to steps S201-S204 in FIG. 2, and the 21-dimensional feature vector of each sliding window is extracted. By using the trained SVM multi-class classifier, the swimming stroke currently used by the user can be identified.

Since the SVM deals with the two-classification problem, when using the SVM to identify multiple swimming strokes, a classifier must be constructed. The present disclosure adopts a “one-to-one” method. In other words, any two types of training samples are selected from the N classes to train one two-class classifier, and a total of N*(N−1)/2 two-class classifiers are needed. Although this method needs a large quantity of classifiers, the accuracy is high. The test sample is inputted into the SVM classifier, and the final identifying result is generated by using a max-wins-voting (MWV) strategy.

For example, the template data of standard swimming states include four types: breaststroke, freestyle, backstroke, and butterfly. In order to identify the user's turn-back action during swimming, in the present embodiment, the turn-back action is also taken as a type of swimming state. In other word, in the present embodiment, there are five types of swimming state, and there are two implementing ways in the SVM training.

The first way is to select any two types of training samples to train one two-class classifier, and a total of N*(N−1)/2 two-class classifiers are needed. In an example, assuming that N=4, and breaststroke and backstroke are selected to form one two-class classifier, namely, a breaststroke/backstroke classifier. Then test datum A is substituted into the formula of the optimal classification function of the two-class classifier, and a matching result about whether the test datum A is a breaststroke or a backstroke can be obtained. Then, the test datum A is compared with the remaining five two-class classifiers (swimming types) respectively, and five matching results are obtained. Finally, based on the quantity of occurrences of the template data in the matching results of the test datum A, the template data with the most quantity of occurrences is taken as the template data that match the test datum A, i.e., the type to which the test datum A belongs. In this example, after comparing the test datum A with six SVM two-class classifiers, it was found that the breaststroke appeared three times, and the backstroke, freestyle and butterfly strokes each appeared once, so the test datum A was identified as a breaststroke.

The second way is that, when training the SVM two-class classifier, in order to reduce the quantity of comparisons, one type of swimming stroke may be regarded as the first type, and all of the other types than this swimming stroke may be regarded as the second type. Thus, in the present embodiment, in order to identify swimming strokes, the identifying result can be obtained by comparing with the SVM two-class classifiers only for four times. For example, in an SVM two-class classifier, breaststroke is taken as the first type, and all of the other types than breaststroke (i.e., freestyle, backstroke, butterfly, and turn-back action) are taken as the second type. In actual applications, the test data are matched with this SVM two-class classifier. After the first comparison, it can be determined whether the test data are a breaststroke. If it is not a breaststroke, the test data are further compared with other trained SVM two-class classifiers, and the specific type of the test data can be determined.

Step 36, turn-back point identification.

If the position of the turn-back point of the user in swimming is identified, the quantity of the trips that the user has swum can be further counted, and parameters such as the speed of swimming can be further calculated according to the length of the pool. However, the turn-back habits and strokes of different persons are very different, so the occurrence of the turn-back action cannot be reliably identified merely by acceleration threshold judgment and other methods, since the acceleration data may change abruptly when turning around.

One solution is to judge the slope change of the acceleration curve. If the slope suddenly increases and exceeds a certain threshold, it is determined that the turn-back action occurs. Another solution is to judge the magnitude of the acceleration. If the amplitude suddenly increases and exceeds a certain threshold, it is determined that the turn-back action occurs. However, those methods are not reliable, because for different swimming strokes and different persons, the turn-back actions vary greatly, and the situations are very different. Sometimes the acceleration changes do not conform to these rules, so it is not reliable to judge simply by the acceleration threshold.

In order to effectively identify the turn-back action, in the present disclosure, the turn-back action is also identified as one type of swimming state, and is trained and identified using the above methods together with the four basic swimming strokes, so a total of five types of patterns need to be identified. Moreover, in order to further reduce the misidentification of the turn-back action (i.e., a turn-back action is identified but there isn't a turn-back action), time tracking is started after a turn-back action is identified, and the turn-back action will occur again only after a time threshold TH_T is exceeded. The reason is that there is a minimum time interval TH_T for each swim trip, and the next turn-back action will not occur in the minimum time interval after a turn-back was determined. If a turn-back action is detected within this minimum time interval, it will be disregarded directly.

### Fourth Embodiment

A wearable device is provided in the present embodiment. FIG. 5 is a structural block diagram of a wearable device according to an embodiment of the present disclosure. Referring to FIG. 5, a sensor is provided in the wearable device, and the wearable device 50 comprises:

a data collecting unit 501, for controlling the sensor to collect movement data of a user when a monitoring process starts;

a feature extracting unit 502, for extracting one or more features for identifying the movement state of the user from the movement data to obtain test data; and

a state monitoring unit 503, for matching the test data with stored template data representing predetermined movement states, to obtain template data that successfully match the test data, and determining occurrence of a movement state corresponding to the matched template data associated with the test data.

In an embodiment of the present disclosure, the data collecting unit 501 is for controlling the sensor to collect movement data of the user of one or more axis directions; and

the feature extracting unit 502 is for extracting one or more of the following time domain features from the movement data of each axis direction: mean value, standard deviation, minimum, maximum, skewness, kurtosis and correlation coefficient.

In an embodiment of the present disclosure, the wearable device is for monitoring the swimming movement state of the user, and controlling a sensor to collect swimming action data of a user when a monitoring process starts; extracting one or more features for identifying a swimming state of the user from the swimming action data to obtain test data; matching the test data with each template data representing a swimming movement state, to obtain a template data that successfully match the test data, and identifying the swimming state of the user as a swimming state corresponding to the template data associated with the test data;

The template data are generated by using collected standard swimming state data of a plurality of users and stored in the wearable device. The standard swimming state data include at least the following types of data: breaststroke data, freestyle data, butterfly data, backstroke data, and turn-back state data.

Identifying the swimming state of the user as a swimming state corresponding to the template data associated with the test data comprises: identifying the swimming state of the user as breaststroke swimming stroke, freestyle swimming stroke, butterfly swimming stroke, backstroke swimming stroke or turn-back state corresponding to the template data associated with the test data.

In an embodiment of the present disclosure, a three-axis acceleration sensor is controlled to collect three-axis acceleration data of the user when he/she is swimming, and the collected three-axis acceleration data is saved in a buffer. Prior to the step of extracting one or more features for identifying a swimming state of the user from the swimming action data, the following preprocessing operations are performed on the collected three-axis acceleration data:

sampling simultaneously from the buffer according to a preset frequency, and performing windowing processing on the sampled data by using a sliding window of a preset step length to obtain acceleration data of each axis direction having a predetermined length, wherein the moving step length of the sliding window needs to satisfy the condition that data of adjoining sliding windows partially overlap; and

performing smoothing filtering on the obtained acceleration data of each axis direction having the predetermined length by using K time-nearest neighbor mean filtering respectively to remove interference noise.

In an embodiment of the present disclosure, the wearable device further comprises: a dimension reducing unit, for calculating correlation between test data consisting of one or more features and movement state of the user by using statistical analysis, screening the test data according to the correlation between the test data and the movement state of the user, to obtain test data after screened, and matching the test data after screened with the template data.

In an embodiment of the present disclosure, the state monitoring unit is for:

training SVM (support vector machine) classifiers by using the template data, for each SVM classifier selecting any two types of template data from the template data to train a two-class classifier, and obtaining a trained SVM two-class classifier which can distinguish any two types of template data in N types of template data; and

matching the test data with each trained SVM two-class classifier respectively, to obtain matching results between the test data and each SVM two-class classifier, wherein each matching result corresponding to one piece of the template data, counting up a quantity of occurrences of the template data, and taking the template data with the most quantity of occurrences as the template data that successfully match the test data.

In an embodiment of the present disclosure, the wearable device further comprises: a turn-back action confirming unit, for after determining that the current swimming state of the user is the turn-back state, judging whether a time interval between a time point of occurrence of a current turn-back state and a time point of occurrence of a previous turn-back state is greater than a preset time threshold, and if yes, determining the current turn-back state to be judged as valid, if not, determining the current turn-back state to be judged as invalid; and when the turn-back state is judged as valid, saving the time point of occurrence of the current turn-back state, and updating the stored time point of occurrence of turn-back state with the time point of occurrence of the current turn-back state.

In an embodiment of the present disclosure, the wearable device further comprises: a static determining unit, for prior to matching the test data with each template data, calculating standard deviations of the collected sensor data on each axis respectively; and comparing the standard deviations of the sensor data on each axis with a preset standard deviation threshold, and if all of the standard deviations of the sensor data on each axis are less than the standard deviation threshold, determining that the user is not in a moving state and not performing further matching processing.

It should be noted that the wearable device of the present embodiment may be applied to the above method for monitoring a movement state by using a wearable device. More details of the working process of the wearable device in the present embodiment may be referred to the description of the method for monitoring a movement state by using a wearable device in the above embodiments, which will not be repeated here.

### Fifth Embodiment

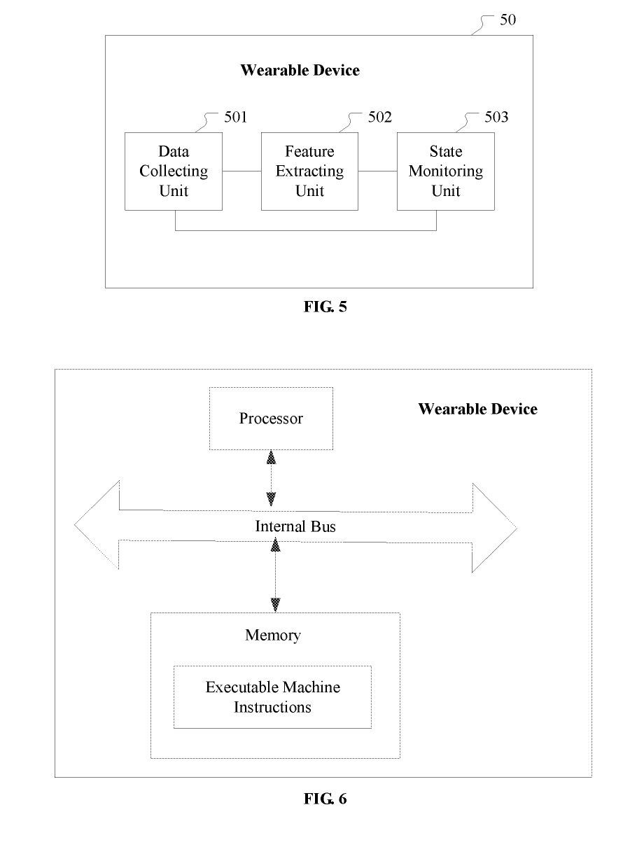

A wearable device is provided in the present embodiment. FIG. 6 is a schematic diagram of the hardware structure of a wearable device according to still another embodiment of the present disclosure. As shown in FIG. 6, the wearable device comprises a processor and a memory that are communicatively connected by an internal bus. The memory stores machine instructions that are executable by the processor, and when executed by the processor, the machine instructions can implement the steps of the method as stated above.

In sum, compared with the prior art such as using video analysis or professional detection equipment, by using an acceleration sensor built in the smart watch, the technical solutions of the present disclosure are convenient to carry, flexible to use, and can identify swimming strokes in real time, which makes it convenient for the user to know his movement state and statistics at any time.

Moreover, in order to adapt to the limited source of a wearable device such as a smart watch, in the present disclosure, only a limited quantity of typical and truly distinguishable time domain features are extracted. Compared with extracting other frequency domain or time-frequency domain features, complicated feature calculations can be avoided, and the amount of calculation can be reduced.

In addition, the use of support vector machine (SVM) with good generalization ability in identifying can provide non-user-restricted identifying ability. In other words, the swimming strokes of different users can be well identified, and the individual training for every user before use is not needed anymore, which facilitates the use and improves the user experience.

Furthermore, by adopting a technical means combining the feature extraction and template data matching, the technical solutions of the present disclosure can identify a plurality of movement states of the user, and it is proved by experimentation that the accuracy of the monitoring results obtained by the technical solutions of the present disclosure is high.

The above merely describes particular embodiments of the present disclosure. By the teaching of the present disclosure, a person skilled in the art can make other modifications or variations based on the above embodiments. A person skilled in the art should appreciate that, the detailed description above is only for the purpose of better explaining the present disclosure, and the protection scope of the present disclosure should be subject to the protection scope of the claims.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment, it being understood that various changes may be made in the function and arrangement of elements described in an exemplary embodiment without departing from the scope of the invention as set forth in the appended claims and their legal equivalents.

1. A method for monitoring a movement state by using a wearable device, wherein a sensor is provided in the wearable device, and the method comprises:
controlling the sensor to collect movement data of a user when a monitoring process starts;
extracting one or more features for identifying the movement state of the user from the movement data to obtain test data; and
matching the test data with stored template data representing predetermined movement states, to obtain template data that successfully match the test data, and determining occurrence of a movement state corresponding to the matched template data associated with the test data.

2. The method according to claim 1, wherein the step of controlling the sensor to collect movement data of a user comprises:
controlling the sensor to collect movement data of the user of one or more axis directions; and
the step of extracting one or more features for identifying the movement state of the user from the movement data comprises:
extracting one or more of the following time domain features from the movement data of each axis direction: mean value, standard deviation, minimum, maximum, skewness, kurtosis and correlation coefficient.

3. The method according to claim 1, wherein the method comprises:
controlling the sensor to collect swimming action data of the user when a monitoring process starts;
extracting one or more features for identifying a swimming state of the user from the swimming action data to obtain test data; and
matching the test data with each template data representing a swimming movement state, to obtain template data that successfully match the test data, and identifying the swimming state of the user as a swimming state corresponding to the template data associated with the test data;
wherein the template data are generated by using collected standard swimming state data of a plurality of users and stored in the wearable device;
the standard swimming state data include at least the following types of data: breaststroke data, freestyle data, butterfly data, backstroke data, and turn-back state data; and
the step of identifying the swimming state of the user as a swimming state corresponding to the template data associated with the test data comprises: identifying the swimming state of the user as breaststroke swimming stroke, freestyle swimming stroke, butterfly swimming stroke, backstroke swimming stroke or turn-back state corresponding to the template data associated with the test data.

4. The method according to claim 3, wherein the step of controlling the sensor to collect swimming action data of the user comprises:
controlling a three-axis acceleration sensor to collect three-axis acceleration data of the user when he/she is swimming, and saving the collected three-axis acceleration data in a buffer; and
prior to the step of extracting one or more features for identifying a swimming state of the user from the swimming action data, performing the following preprocessing operation on the collected three-axis acceleration data:
sampling simultaneously from the buffer according to a preset frequency, and performing windowing processing on the sampled data by using a sliding window of a preset step length to obtain acceleration data of each axis direction having a predetermined length, wherein the moving step length of the sliding window satisfies the condition that data of adjoining sliding windows partially overlap; and
performing smoothing filtering on the obtained acceleration data of each axis direction having the predetermined length by using K time-nearest neighbor mean filtering respectively to remove interference noise.

5. The method according to claim 1, further comprising:
calculating correlation between test data consisting of one or more features and movement state of the user by using statistical analysis, screening the test data according to the correlation between the test data and the movement state of the user, to obtain test data after screened, and matching the test data after screened with the template data.

6. The method according to claim 1, wherein the step of matching the test data with stored template data representing predetermined movement states, to obtain template data that successfully match the test data, and determining occurrence of a movement state corresponding to the matched template data associated with the test data comprises:
training SVM (Support Vector Machine) classifiers by using the template data, for each SVM classifier selecting any two types of template data from the template data to train a two-class classifier, and obtaining a trained SVM two-class classifier which can distinguish any two types of template data in N types of template data; and
matching the test data with each trained SVM two-class classifier respectively, to obtain matching results between the test data and each SVM two-class classifier, wherein each matching result corresponding to one piece of the template data, counting up a quantity of occurrences of the template data, and taking the template data with the most quantity of occurrences as the template data that successfully match the test data.

7. The method according to claim 3, further comprising:
after determining that the current swimming state of the user is the turn-back state, judging whether a time interval between a time point of occurrence of a current turn-back state and a time point of occurrence of a previous turn-back state is greater than a preset time threshold, and if yes, determining the current turn-back state to be judged as valid, if not, determining the current turn-back state to be judged as invalid; and
when the turn-back state is judged as valid, saving the time point of occurrence of the current turn-back state, and updating the stored time point of occurrence of turn-back state with the time point of occurrence of the current turn-back state.

8. The method according to claim 2, prior to the step of matching the test data with each template data, the method further comprises:
calculating standard deviations of the collected sensor data on each axis respectively; and
comparing the standard deviations of the sensor data on each axis with a preset standard deviation threshold, and if all of the standard deviations of the sensor data on each axis are less than the standard deviation threshold, determining that the user is not in a moving state and not performing further matching processing.

9. A wearable device, wherein a sensor is provided in the wearable device, and the wearable device comprises:
a data collecting unit, for controlling the sensor to collect movement data of a user when a monitoring process starts;
a feature extracting unit, for extracting one or more features for identifying the movement state of the user from the movement data to obtain test data; and
a state monitoring unit, for matching the test data with stored template data representing predetermined movement states, to obtain template data that successfully match the test data, and determining occurrence of a movement state corresponding to the matched template data associated with the test data.

10. The wearable device according to claim 9, wherein
the data collecting unit is for controlling the sensor to collect movement data of the user of one or more axis directions; and
the feature extracting unit is for extracting one or more of the following time domain features from the movement data of each axis direction: mean value, standard deviation, minimum, maximum, skewness, kurtosis and correlation coefficient.

11. The wearable device according to claim 9, wherein
the data collecting unit is for controlling the sensor to collect swimming action data of the user when a monitoring process starts;
the feature extracting unit is for extracting one or more features for identifying a swimming state of the user from the swimming action data to obtain test data;
the state monitoring unit is for matching the test data with each template data representing a swimming movement state, to obtain template data that successfully match the test data, and identifying the swimming state of the user as a swimming state corresponding to the template data associated with the test data;
wherein the template data are generated by using collected standard swimming state data of a plurality of users and stored in the wearable device;
the standard swimming state data include at least the following types of data: breaststroke data, freestyle data, butterfly data, backstroke data, and turn-back state data; and
the state monitoring unit is for identifying the swimming state of the user as breaststroke swimming stroke, freestyle swimming stroke, butterfly swimming stroke, backstroke swimming stroke or turn-back state corresponding to the template data associated with the test data.

12. The wearable device according to claim 11, wherein the data collecting unit is for controlling a three-axis acceleration sensor to collect three-axis acceleration data of the user when he/she is swimming, and saving the collected three-axis acceleration data in a buffer; and
prior to the extracting one or more features for identifying a swimming state of the user from the swimming action data, performing the following preprocessing operation on the collected three-axis acceleration data:
sampling simultaneously from the buffer according to a preset frequency, and performing windowing processing on the sampled data by using a sliding window of a preset step length to obtain acceleration data of each axis direction having a predetermined length, wherein the moving step length of the sliding window satisfies the condition that data of adjoining sliding windows partially overlap; and
performing smoothing filtering on the obtained acceleration data of each axis direction having the predetermined length by using K time-nearest neighbor mean filtering respectively to remove interference noise.

13. The wearable device according to claim 9, further comprising:
a dimension reducing unit, for calculating correlation between test data consisting of one or more features and movement state of the user by using statistical analysis, screening the test data according to the correlation between the test data and the movement state of the user, to obtain test data after screened, and matching the test data after screened with the template data.

14. The wearable device according to claim 9, wherein the state monitoring unit is for:
training SVM (support vector machine) classifiers by using the template data, for each SVM classifier selecting any two types of template data from the template data to train a two-class classifier, and obtaining a trained SVM two-class classifier which can distinguish any two types of template data in N types of template data; and
matching the test data with each trained SVM two-class classifier respectively, to obtain matching results between the test data and each SVM two-class classifier, wherein each matching result corresponding to one piece of the template data, counting up a quantity of occurrences of the template data, and taking the template data with the most quantity of occurrences as the template data that successfully match the test data.

15. The wearable device according to claim 11, further comprising:
a turn-back action confirming unit, for after determining that the current swimming state of the user is the turn-back state, judging whether a time interval between a time point of occurrence of a current turn-back state and a time point of occurrence of a previous turn-back state is greater than a preset time threshold, and if yes, determining the current turn-back state to be judged as valid, if not, determining the current turn-back state to be judged as invalid; and when the turn-back state is judged as valid, saving the time point of occurrence of the current turn-back state, and updating the stored time point of occurrence of turn-back state with the time point of occurrence of the current turn-back state.

16. The wearable device according to claim 10, further comprising:
a static determining unit, for prior to matching the test data with each template data, calculating standard deviations of the collected sensor data on each axis respectively; and comparing the standard deviations of the sensor data on each axis with a preset standard deviation threshold, and if all of the standard deviations of the sensor data on each axis are less than the standard deviation threshold, determining that the user is not in a moving state and not performing further matching processing.
